(19) United States
(12) Patent Application Publication
(10) Pub. No.: US 2025/0170397 A1
(43) Pub. Date: May 29, 2025

(54) PAIN RELIEF DEVICE AND CONTROL METHOD THEREOF

(71) Applicant: LEE SOL CO., LTD., Seoul (KR)

(72) Inventors: Seung Woo LEE, Seoul (KR); Gusung KWON, Seoul (KR)

(21) Appl. No.: 18/746,089

(22) Filed: Jun. 18, 2024

(30) Foreign Application Priority Data
Nov. 28, 2023 (KR) 10-2023-0167941

(51) Int. Cl. A61N 1/36

(52) U.S. Cl. CPC A61N 1/36021; A61N 1/36034

(57) ABSTRACT

A pain relief device includes a generating unit that generates an electrical signal recognized as non-painful by the brain; an electrode attached to at least one part of the body to deliver the generated electrical signal; and a control unit that controls the operation of the generating unit and the electrode, and, during the process in which a pain signal generated in a first region of the body is transmitted to the brain, the electrical signal delivered by the electrode is transmitted to the brain along with the pain signal through surface stimulus receptor of the first region, thereby alleviating the pain perceived by the brain in the first region, and the electrical signal is an alternating current stimulation (ACS) signal, and the alternating current stimulation signal repeatedly switches on and off according to a predetermined first frequency, and may be a first combined stimulation that applies the signal switched on according to the first frequency as stimuli according to a predetermined second frequency.

### CROSS-REFERENCE TO RELATED APPLICATIONS

This application claims the priority to Korean Patent Application No. 10-2023-0167941 filed on Nov. 28, 2023, in the Korean Intellectual Property Office, the disclosure of which is incorporated herein by reference.

### BACKGROUND

### Field

The present invention relates to a pain relief device and a control method thereof, and more specifically, relates to a pain relief device and a control method that transmit an electric signal which is recognized as painless by the brain, along with a pain signal generated from a part of the body to the brain, thereby causing the user's brain to mistakenly perceive that the pain signal is not generated, while simultaneously improving blood flow and alleviating pain generated from a part of the body.

### Description of the Related Art

Pain is a defensive mechanism of the human body, where nerve endings are stimulated, resulting in discomfort, anxiety, or painful localized sensations, which act to avoid or remove damage. Meanwhile, pain can be seen as an abstract and subjective concept, essentially a very personal sensation. The classification committee of the International Association for the Study of Pain (IASP) defined pain as an unpleasant sensory and emotional experience associated with actual or potential tissue damage, that is, pain is an indispensable sense for animal survival and a complex biological response, which appears as the first sign in the body when a disease occurs. However, The IASP explained, in chronic cases, pain itself can be regarded as a disease. The IASP further classifies pain by location, system, duration, intensity, and cause, but it is generally classified by duration. Typically, pain lasting more than 3 months is classified as chronic pain.

Pain can be caused not only by physical factors but also by mental and social factors. Treatment methods vary, including pharmacotherapy, physical therapy, surgical treatment, and psychotherapy.

Among the above treatment methods for pain, pharmacotherapy often leads to increasing dosage and frequency of drug administration due to drug tolerance. This can result in many side effects and high costs associated with long-term drug use. Other treatment methods also have the problem of being temporary or expensive.

To improve the above pain treatment methods, the applicant proposes a pain scrambler.

The pain scrambler is a device that controls pain by mixing a calculated electrical signal during the process where a pain signal caused by pain occurring in a part of the user's body is transmitted (or applied) to the brain (central nervous system), thereby preventing the user from feeling pain. The electrical signal generated by the pain scrambler is a fine current similar to a biological signal in the body, making the user's brain perceive them as non-painful. This creates an illusion in the user's brain that no pain signal is being generated. Additionally, by increasing ATP (adenosine triphosphate) in the cells where pain occurs, it improves blood flow and alleviates inflammation (or pain).

### SUMMARY

Therefore, the present invention has been devised to solve the aforementioned problems. The object of the present invention is to provide a pain relief device and a control method thereof, which deliver an electrical signal recognized as non-painful in the brain, along with a pain signal generated from a part of the body, to the brain, thereby causing the user's brain to mistakenly perceive that no pain signal is being generated, while simultaneously alleviating the pain occurring in a part of the body.

However, the technical objects to be achieved by the present invention are not limited to the aforementioned technical objects. Other technical objects, which are not mentioned above, will be apparently understood to a person having ordinary skill in the art from the following description.

A pain relief device according to an exemplary embodiment of the present invention for achieving the above objects comprises: a generating unit that generates an electrical signal recognized as non-painful by the brain; an electrode attached to at least a part of the body to deliver the generated electrical signal; and a control unit that controls the operation of the generating unit and the electrode, wherein the electrical signal delivered by the electrode is transmitted to the brain along with the pain signal through surface stimulus receptor in the first region during the process where the pain signal generated in a first region of the body is transmitted to the brain, thereby alleviating the pain perceived by the brain in the first region, wherein the electrical signal is alternating current stimulation (ACS) signals, wherein the alternating current stimulation signal repeatedly switches on and off according to a predetermined first frequency, and is a first combined stimulation that applies the signal switched on according to the first frequency as stimuli according to a predetermined second frequency.

In addition, a control method of a pain relief device according to an embodiment of the present invention for achieving the above object comprises: a generating unit that generates an electrical signal recognized as non-painful by the brain; an electrode attached to at least a part of the body to deliver the generated electrical signal; and a control unit that controls the operation of the generating unit and the electrode, and the control method of the pain relief device comprises: a) a first step of delivering the electrical signal to a surface stimulus receptor in a first region of the body; b) a second step in which the electrical signal delivered to the surface stimulus receptor in the first region mix with the pain signal during the process where the pain signal generated in the first region is transmitted to the brain; c) a third step in which the electrical signal and the pain signal are transmitted together to the brain; and d) a fourth step in which the pain in the first region perceived by the brain is alleviated by the electrical signal, wherein the electrical signal is an ACS (alternating current stimulation) signal, wherein the alternating current stimulation signal repeatedly switches on and off according to a predetermined first frequency, and is a first combined stimulation that applies the signal switched on according to the first frequency as stimuli according to a predetermined second frequency.

According to one embodiment of the present invention, it is possible to make the user's brain mistakenly perceive that no pain signal has occurred, while simultaneously alleviating the pain generated from the part of the body, by delivering an electric signal to the user's brain, which is recognized as non-painful by the user's brain, mixed with a pain signal generated from a part of the body.

According to one embodiment of the present invention, it is possible to treat the pain area based on alleviating the pain generated from a part of the body.

However, the effects which may be obtained in the present invention are not limited to the aforementioned effects, and other unmentioned effects will be clearly understood by those skilled in the art from the following description.

### BRIEF DESCRIPTION OF THE DRAWINGS

FIGS. 1 to 3 are diagrams illustrating the devices for implementing a pain relief device according to an embodiment of the present invention.

FIG. 4 is a diagram illustrating an example of the electrical signal generated by the generating unit shown in FIG. 1.

FIG. 5 is a diagram illustrating a modified example of the pain relief device.

FIG. 6 is a flowchart illustrating the control method of the pain relief device according to an embodiment of the present invention.

### DETAILED DESCRIPTION OF THE PREFERRED EMBODIMENT

Hereinafter, embodiments of the present invention will be described in detail so as to be easily implemented by those skilled in the art, with reference to the accompanying drawings. However, the description of the present invention is merely an exemplary embodiment for structural or functional description and the scope of the present invention should not be construed as being limited by exemplary embodiments described in a text. That is, since the exemplary embodiment may be variously changed and have various forms, the scope of the present invention should be understood to include equivalents capable of realizing the technical idea. Further, since the objects or effects presented in the present invention are not intended to mean that all of them must be included in a specific embodiment or that only such effects should be included, the scope of the present invention should not be understood as being limited by the object or effect.

Meanings of the terms described in the present invention should be understood as follows.

The terms “first”, “second”, and the like are used to differentiate one component from other components, but the scope should not be construed to be limited by the terms. For example, a first component may be referred to as a second component, and similarly, the second component may be referred to as the first component. It should be understood that, when it is described that a component is “connected to” the other component, the component may be directly connected, or another component may be present therebetween. In contrast, it should be understood that, when it is described, a component is “directly connected to” the other component, another component is not present therebetween. Meanwhile, other expressions describing the relationships between components, such as “between” and “directly between” or “adjacent to” and “directly adjacent to” should be similarly interpreted.

It should be understood that singular expressions encompass a plurality of expressions unless the context clearly dictates otherwise and it should be understood that the terms like “comprise” or “have” indicate a feature, a number, a step, an operation, a component, a part or the combination thereof described in the specification is present, but do not exclude a possibility of the presence or addition of one or more other features, numbers, steps, operations, components, parts, or combinations thereof, in advance.

If it is not contrarily defined, all terms used herein have the same meanings as those generally understood by those skilled in the art to which the present invention belongs. Terms which are defined in generally used dictionary should be interpreted to have the same meanings as the meaning in context of the related art, and are not be interpreted as an ideal meaning or excessively formal meanings unless clearly defined in the present invention.

### Pain Relief Device

A pain relief device (10) according to an embodiment of the present invention is a pain scrambler that controls pain by preventing the user from feeling pain by mixing a calculated electrical signal during the process where a pain signal caused by pain occurring in a part of the user's body is transmitted (or applied) to the brain (central nervous system).

The electrical signal (100) generated by the pain relief device (10) of the present invention is a fine current similar to a biological signal in the body, making the user's brain perceive them as non-painful, and this creates an illusion in the user's brain that no pain signal is being generated. In addition, by increasing ATP (adenosine triphosphate) in the cells where pain occurs, it improves blood flow and alleviates inflammation (or pain).

Further, the pain relief device (10) of the present invention may enable treatment of the pain area based on alleviating pain occurring in a part of the body through the electrical signal (100).

In the present invention, the types of pain occurring in a part of the user's body are not limited. In one embodiment, it refers to knee joint pain, frozen shoulder pain, shoulder impingement syndrome pain, carpal tunnel syndrome pain, plantar fasciitis pain, rheumatoid arthritis pain, postherpetic neuralgia pain, back pain, lumbar stenosis pain, neck pain, and so on.

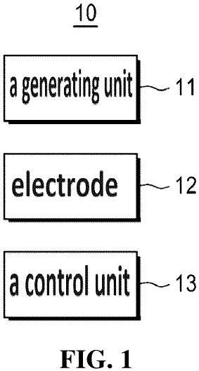

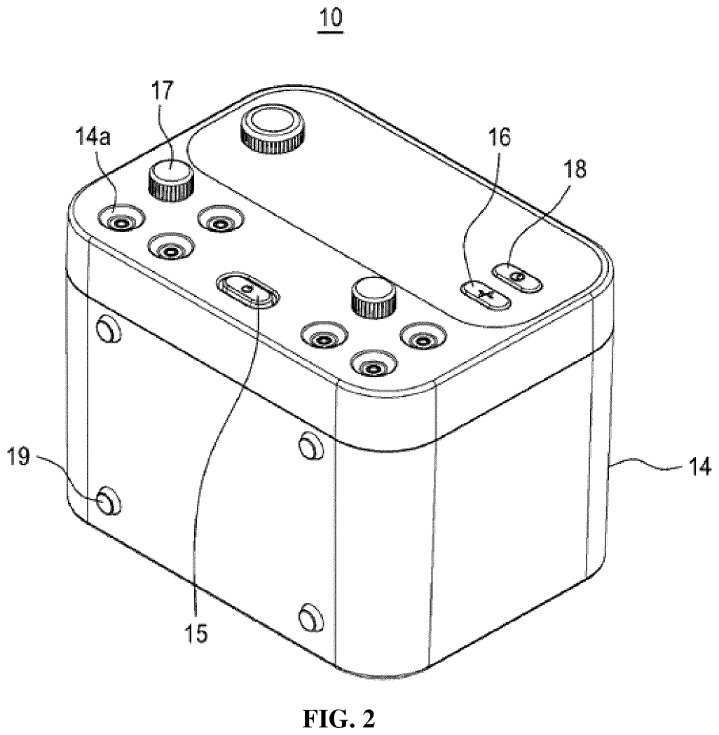

Meanwhile, to make the user's brain mistakenly perceive that no pain signal is being generated while simultaneously improving blood flow and alleviating inflammation, the pain relief device (10) may be implemented through the devices shown in FIGS. 1 and 2 according to one embodiment.

FIGS. 1 to 3 illustrate the devices for implementing the pain relief device according to an embodiment of the present invention.

Referring to FIGS. 1 to 3, the pain relief device (10) comprises a generating unit (11), an electrode (12), a control unit (13), a main body (14), a power unit (15), a stimulus adjustment unit (16), an intensity control unit (17), a timer setting unit (18), and rubber packing (19).

The generating unit (11) may generate an electrical signal (100) based on a fine current similar to a biological signal in the body, which will be perceived as non-painful by the user's brain.

In one embodiment, the generating unit (11) is disposed in the internal space of the main body (14) (not shown in the Figures) and may deliver the generated an electrical signal (100) to the electrode (12) inserted into the electrode insertion part (14a) of the main body (14).

The electrode (12), when it attaches to a first region of the user's body where pain occurs, may deliver the electrical signal (100) received from the generating unit (11) to the surface stimulus receptor of the first region, thereby transmitting them to the user's brain.

In one embodiment, the electrode (12) should be placed in the first region. where a pain signal are generated and may have a certain degree of adhesiveness to allow for attachment and detachment in the placed state. The electrode (12) may be provided in multiples to ensure that pain relief in the first region or simultaneous pain relief in various body regions including the first region is achieved regardless of the size of the first region.

In addition, when multiple electrodes (12) are inserted into the electrode insertion part (14a) provided on one side of the main body (14), they receive an electrical signal from the generating unit (11) through the control unit (13) and can deliver the received electrical signal (100) to the surface stimulus receptor of the first region.

Further, the multiple electrodes (12) deliver electrical signals to the surface stimulus receptor of the first region to promote the generation of ATP (adenosine triphosphate) in the first region. Based on the promoted ATP generation, blood flow improvement may be achieved, thereby improving inflammation related to the first region.

The electrical signal (100) to be delivered to the surface stimulus receptor of the first region from the electrode (12) refers to a signal with an current value similar to a biological signal within a certain error range of the body, enabling the user's brain to perceive them as non-painful. This ensures the pain relief device (10) functions as a pain scrambler.

Additionally, the electrical signal (100) refers to a current with an intensity ranging from 100 μA to 1000 μA to be perceived as non-painful by the user's brain.

Moreover, the electrical signals (100) may be generated in multiples by the generating unit (11) to alleviate pain in the first region, and they may be delivered to the surface stimulus receptor of the first region for the duration set by the timer setting unit (18) and then transmitted to the user's brain.

It is desirable that these electrical signals (100) be understood as non-painful signals delivered to the surface stimulus receptor of the first region. By receiving the electrical signals (100), which are non-painful, along with the pain signals generated in the first region, the user's brain perceives the condition of the first region as non-painful.

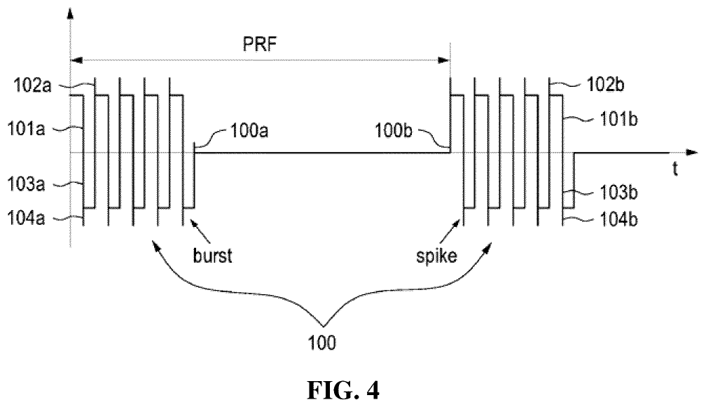

Further, as shown in FIG. 4, the electrical signal (100) may be an alternating current stimulation (ACS) signal, represented on a graph with time on the x-axis and a current value on the y-axis.

FIG. 4 illustrates an example of the electrical signal generated by the generating unit shown in FIG. 1.

Referring to FIG. 4, the electrical signal (100) of the present invention is repeatedly generated for the duration set by the timer setting unit (18) and can be delivered to the surface stimulus receptor of the first region where pain signal occur through multiple electrodes (12).

In the present invention, multiple electrical signals (100) are not limited. However, in the following embodiment, the description will be based on the first electrical signal (100a) and the second electrical signal (100b) shown in FIG. 4.

In one embodiment, the multiple electrical signal (100a, 100b) repeatedly switches on and off according to a predetermined first frequency, which refers to a burst signal.

In this context, a burst signal refers to a series of a limited number of unique pulses or vibrations of limited duration.

In addition, the burst signal may repeatedly include a positive signal having a value exceeding a predetermined standard and a negative signal having a value below the predetermined standard.

As shown in FIG. 4 in one embodiment, the burst signal refers to a combined rectangular signal (101, 103) that includes multiple signals with a positive and a negative current value.

Among the multiple rectangular signals, the first rectangular signal (101) with a positive current value may consist of a first rectangular signal (101a) and a second rectangular signal (101b) in one embodiment, corresponding to the first and second electrical signals (100a, 100b).

The first rectangular signals (101a, 101b) may be implemented as rectangular burst signals with identical widths on the x-axis (time) and identical lengths on the y-axis (current value) when the maximum positive current value is set the same for the electrical signal (100) generated by the generating unit (11).

These first rectangular signals (101a, 101b) may include at least one positive spike signal (102).

The positive spike signal (102) is preferably composed of multiple signals, corresponding to the first and second electrical signals (100a, 100b). In one embodiment, it may consist of a first positive spike signal (102a) and a second positive spike signal (102b).

In addition, the positive spike signal (102) may have a regular pattern on the first rectangular signal (101) and may occur at equal time intervals when the electrical signal (100) is generated by the generating unit (11).

Meanwhile, among the multiple rectangular signals, the second rectangular signal (103) with a negative current value may consist of a second rectangular signal (103a) and a second rectangular signal (103b) in one embodiment, corresponding that the electrical signal (100) is the first and second electrical signals (100a, 100b).

In addition, the second rectangular signals (103a, 103b) may be implemented as rectangular burst signals with identical widths on the x-axis (time) and identical lengths on the y-axis (current value) when the maximum negative current value is set the same for the electrical signal (100) generated by the generating unit (11).

These second rectangular signals (103a, 103b) may include at least one negative spike signal (104).

The negative spike signal (104) is preferably composed of multiple signals, corresponding that the electrical signal (100) is the first and second electrical signals (100a, 100b). In one embodiment, it may consist of a first negative spike signal (104a) and a second negative spike signal (104b).

Additionally, the negative spike signal (104) may have a regular pattern on the second rectangular signal (103) and may occur at equal time intervals when the electrical signal (100) is generated by the generating unit (11).

In one embodiment, the multiple electrical signals (100a, 100b) may be the first combined stimulation where the signals turned on according to the first frequency are applied as stimuli according to a predetermined second frequency, and the second frequency may refer to the pulse repetition frequency (PRF).

The pulse repetition frequency refers to the number of pulses of the electrical signals (100a, 100b) repeated within a specific time unit.

As shown in FIG. 4, the second frequency, which is the pulse repetition frequency, includes a rest period without pulses after the burst signal and a rectangular signal (101, 103) which is the burst signal. The rest period of the burst is a critical feature because, without it, the user's brain may not perceive the electrical signal (100).

The electrical signals (100a, 100b) are shown to enter the rest period after passing through the last second rectangular signal (103) in FIG. 4, but this is not limited, and they may enter the rest period after the last first rectangular signal (103).

Referring again to FIG. 1, the control unit (13) may control the operation of the generating unit (11) and the electrode (12).

In one embodiment, the control unit (13) may be disposed in the internal space of the main body (14) (not shown in the drawings) and may control the operation of the generating unit (11) and the electrode (12).

Specifically, the control unit (13) may control the operation of the generating unit (11) to generate an electrical signal (100) based on a second combined stimulation, which involves modifying at least one of the first frequency (burst), the second frequency (PRF), and the output, the waveform and the period of the stimulus according to the second frequency, when the degree of pain relief in the first region according to the first combined stimulation based on the electrical signal (100) is below a predetermined range.

In addition, the control unit (13) may control the operation of the generating unit (11) so that the electrical signal (100) based on the second combined stimulation is delivered to the multiple electrodes (12). Subsequently, the control unit (13) may control the operation of the multiple electrodes (12) to deliver the electrical signals (100) based on the second combined stimulation to the surface stimulus receptor of the first region.

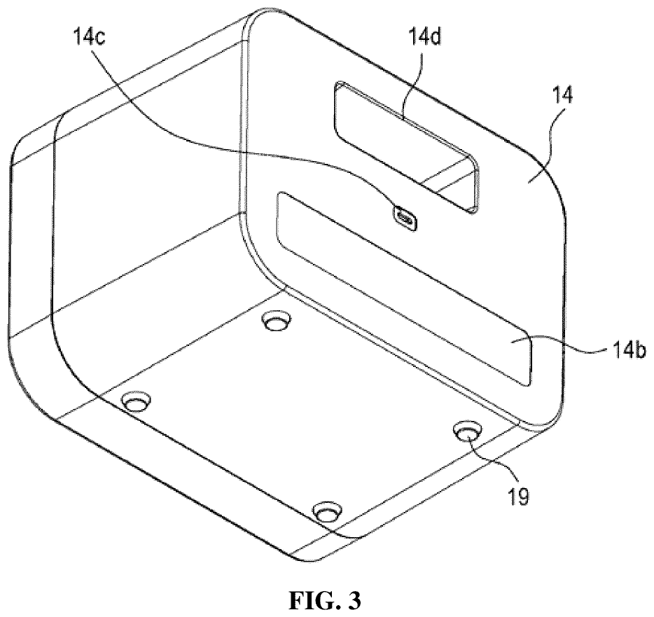

Referring to FIGS. 2 and 3, the main body (14) may include a power unit (15), a stimulus adjustment unit (16), an intensity control unit (17), and a timer setting unit (18) on different positions of one side to allow the user or medical personnel providing the pain relief service to operate the pain relief device (10).

Additionally, the main body (14) may be provided with one or more electrode insertion parts (14a) on one side, into which multiple electrodes (12) can be inserted.

In this case, the electrode insertion part (14a) is preferably provided on one side of the main body (14) in a one-to-one ratio with the maximum number of electrodes (12) that can be provided to the user or medical personnel. For example, if the pain relief device (10) can provide up to six electrodes (12), six electrode insertion parts (14a) may be provided on one side of the main body (14).

The main body (14) may be provided with a battery mounting part (14b) so that a battery (not shown in the Figure), which supplies electrical energy to the generating unit (11) and the control unit (13), can be mounted in the internal space.

Additionally, the main body (14) may be provided with a cable connection part (14c) to connect a cable (not shown in the Figure) for externally charging the battery mounted in the battery mounting part (14b).

Further, the main body (14) may be provided with a grip part (14d) of a size and shape that can be held by hand so that the user or medical personnel can place the pain relief device (10) in the desired position.

Preferably, the main body (14) has the battery mounting part (14b), the cable connection part (14c), and the grip part (14d) on sides other than the side where the rubber packing (19) is attached.

In addition, the main body (14) may have one or more rubber packings (19) attached to the opposite side, which can be the bottom surface, to prevent the main body (14) from moving based on friction with the floor when the device is placed on the ground during use.

The main body (14) may also be provided with a display (not shown) on one side to output data such as the current value of the electrical signal (100) delivered to the surface stimulus receptor of the first region, the usage time of the pain relief device (10), the number of electrodes (12) inserted into the electrode insertion part (14a), and the remaining battery level, as well as real-time changes in this data to the user.

The power unit (15) may be arranged on one side of the main body (14) to turn the power of the pain relief device (10) on and off.

Additionally, the power unit (15) may be implemented in the form of a button that allows the user or medical personnel to turn the power of the pain relief device (10) on and off by inputting a signal through a finger or tool.

In one embodiment, when the power of the pain relief device (10) is turned on through the power unit (15), the generating unit (11) and the control unit (13) may operate based on the electrical energy supplied from the battery.

The stimulus adjustment unit (16) may be arranged on one side of the main body (14) to change the combined stimulation method of the electrical signal (100).

Additionally, the stimulus adjustment unit (16) may be implemented in the form of a button that allows the user or medical personnel to change the combined stimulation method of the electrical signal (100) by inputting a signal through a finger or tool.

In one embodiment, the stimulus adjustment unit (16) may send a first request signal to the control unit (13) to change the electrical signal (100) based on the first combined stimulation being delivered to the surface stimulus receptor of the first region through multiple electrodes (12) to an electrical signal (100) based on the second combined stimulation when a signal is input.

That is, in the present invention, the electrical signal (100) based on the first combined stimulation may be changed to an electrical signal (100) based on the second combined stimulation by the control unit (13) when the degree of pain relief in the first region according to the first combined stimulation is below a predetermined range, or may be changed to an electrical signal (100) based on the second combined stimulation when the user or medical personnel inputs a signal to the stimulus adjustment unit (16).

The stimulus adjustment unit (16) is not limited to changing the electrical signal (100) based on the first combined stimulation to the electrical signal (100) based on the second combined stimulation. It may also send a second request signal to the control unit (13) to change the electrical signal (100) based on the second combined stimulation being delivered to the surface stimulus receptor of the first region through multiple electrodes (12) to an electrical signal (100) based on the first combined stimulation.

The intensity control unit (17) may be arranged on one side of the main body (14) to adjust the current value (intensity) of the electrical signal (100) being delivered to the surface stimulus receptor of the first region through multiple electrodes (12).

Additionally, the intensity control unit (17) may be implemented in the form of a button that allows the user or medical personnel to adjust the current value of the electrical signal (100) being delivered to the surface stimulus receptor of the first region by inputting a signal through a finger or tool.

The current value of the electrical signal (100) adjusted through the intensity control unit (17) is not limited, however, in one embodiment, the intensity control unit (17) is preferably provided on one side of the main body (14) to adjust the current value of the electrical signal (100) to 100 μA to 1000 μA so that the electrical signal (100) may be perceived as non-painful by the user's brain.

The timer setting unit (18) may be arranged on one side of the main body (14) to adjust the duration for which the electrical signal (100) is applied to the surface stimulus receptor of the first region through multiple electrodes (12).

Additionally, the timer setting unit (18) may be implemented in the form of a button that allows the user or medical personnel to adjust the duration for which the electrical signal (100) is applied to the surface stimulus receptor of the first region through multiple electrodes (12) by inputting a signal through a finger or tool.

The duration for which the electrical signal (100) is applied to the surface stimulus receptor of the first region through the timer setting unit (18) is not limited, however, in one embodiment, the timer setting unit (18) is preferably provided on one side of the main body (14) to adjust the duration to 10 to 60 minutes so that the pain in the first region may be alleviated.

The rubber packing (19) is arranged on the opposite side of the main body (14). When the main body (14) is placed on the ground (floor) while the user is receiving pain relief services, it can prevent movement of the main body (14) based on friction with the ground while the electrical signal (100) is being applied to the surface stimulus receptor of the first region.

Preferably, at least one rubber packing (19) is arranged on the opposite side of the main body (14) as shown in FIG. 2 to prevent movement of the main body (14).

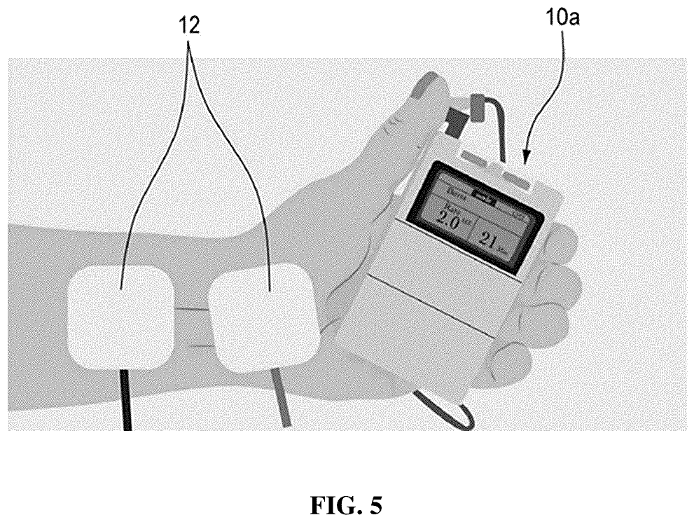

Meanwhile, the pain relief device (10) is not limited to the forms shown in FIGS. 2 and 3, but may be modified to a form of a wearable device (10a) that is portable, smaller, and lighter as shown in FIG. 5, allowing the user or medical personnel to hold it with one hand.

### Control Method of the Pain Relief Device

Hereinafter, the control method (S10) of the pain relief device (10) will be described in detail.

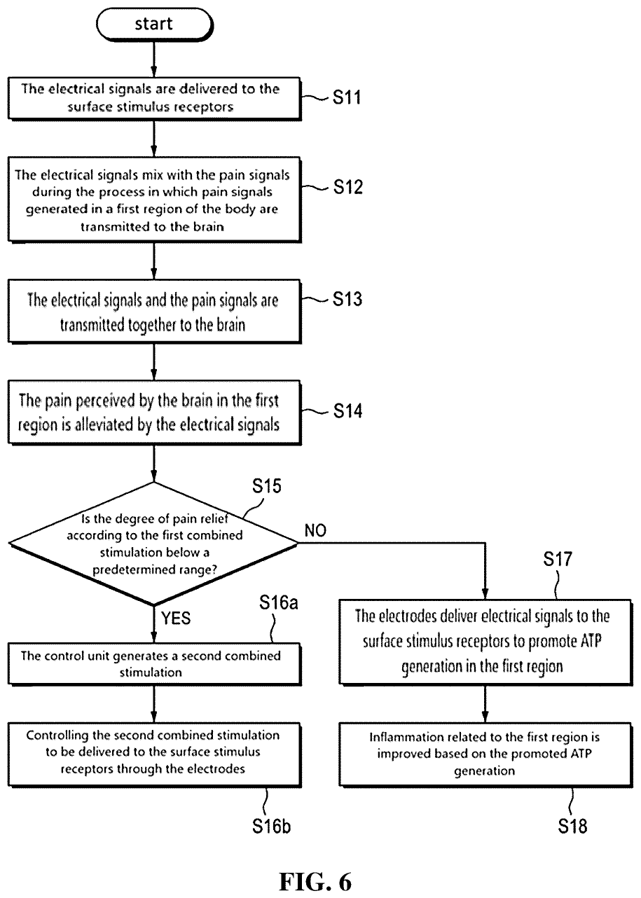

FIG. 6 is a flowchart illustrating the control method of the pain relief device according to an embodiment of the present invention.

Referring to FIG. 6, the control method (S10) of the pain relief device comprises an electrical signal delivery step (S11), a signal mixing step (S12), a signal delivery step (S13), a pain relief step (S14), a pain relief degree judgment step (S15), an electrical signal generation and delivery step based on the second combined stimulation (S16), an electrical signal delivery step based on the first combined stimulation (S17), and an inflammation improvement step (S18).

In the electrical signal delivery step (S11), when the electrical signal (100) based on the first combined stimulation is generated by the generating unit (11), it can be delivered to multiple electrodes (12) from the generating unit (11).

In the signal mixing step (S12), during the process where the pain signal generated in the first region of the body is delivered to the user's brain, the electrical signal (100) may mix with the pain signal.

In the signal delivery step (S13), the electrical signal (100) and the pain signal may be delivered together to the user's brain.

In the pain relief step (S14), the pain perceived by the user's brain in the first region may be alleviated by the electrical signal (100).

In the pain relief degree judgment step (S15), the control unit (13) may judge whether the degree of pain relief according to the first combined stimulation is below a predetermined range due to the electrical signal (100).

If the degree of pain relief according to the first combined stimulation is below the predetermined range (S15-YES), the control unit (13) may control the operation of the generating unit (11) to generate an electrical signal (100) based on the second combined stimulation instead of the electrical signal (100) based on the first combined stimulation (S16a).

Then, the control unit (13) may control the operation of the generating unit (11) and the multiple electrodes (12) so that the electrical signal (100) based on the second combined stimulation is delivered to the surface stimulus receptor of the first region (S16b).

On the other hand, when the degree of pain relief according to the first combined stimulation is within the predetermined range (S15—NO), the control unit (13) may control the operation of the generating unit (11) to continue generating the electrical signal (100) based on the first combined stimulation and control the operation of the multiple electrodes (12) so that the electrical signal (100) based on the first combined stimulation is applied to the surface stimulus receptor of the first region to promote ATP (adenosine triphosphate) generation in the first region (S17).

Subsequently, the multiple electrodes (12) apply the electrical signal (100) based on the first combined stimulation to the surface stimulus receptor of the first region, thereby improving blood flow and alleviating inflammation related to the first region based on the promoted ATP generation (S18).

Meanwhile, the above-described embodiments of the present invention may be implemented through various means. For example, the embodiments of the present invention may be implemented through hardware, firmware, software, or a combination thereof.

In the case of hardware implementation, the methods according to the embodiments of the present invention may be implemented using one or more ASICs (Application Specific Integrated Circuits), DSPs (Digital Signal Processors), DSPDs (Digital Signal Processing Devices), PLDs (Programmable Logic Devices), FPGAs (Field Programmable Gate Arrays), processors, controllers, microcontrollers, microprocessors, etc.

In the case of firmware or software implementation, the methods according to the embodiments of the present invention may be implemented in the form of modules, procedures, or functions that perform the functions or operations described above. The software code may be stored in a memory unit and driven by a processor. The memory unit may be located inside or outside the processor and may exchange data with the processor through various known means.

As described above, the detailed description of the preferred embodiments of the present invention has been provided to enable those skilled in the art to implement and practice the invention. Although the preferred embodiments of the present invention have been described with reference to the above, those skilled in the art will understand that various modifications and changes can be made to the present invention without departing from its scope. For example, those skilled in the art may use each configuration described in the above embodiments in combination. Therefore, the present invention is not intended to be limited to the embodiments described herein but to assign the broadest scope consistent with the disclosed principles and novel features.

The present invention may be embodied in other specific forms without departing from the spirit and essential features of the present invention. Therefore, the detailed description should not be interpreted in a restrictive manner but should be considered as illustrative. The scope of the present invention should be determined by the reasonable interpretation of the appended claims, and all changes within the equivalent scope of the present invention are included. The present invention is not intended to be limited to the embodiments described herein but to assign the broadest scope consistent with the disclosed principles and novel features disclosed. Further, claims having no clear quoting relation in the claims are combined to configure the exemplary embodiment or may be included as new claims by correction after application.

[Explanation of Reference Numerals and Symbols]

| | |
|---|---|
| 10: Pain relief device | 11: Generating unit |
| 12: Electrode | 13: Control unit |
| 14: Main body | 14a: Electrode insertion part |
| 14b: Battery mount | 14c: Cable Connection Part |
| 14d: Grip part | 15: Power unit |
| 16: Stimulus adjustment unit | 17: Intensity control unit |
| 18: Timer setting unit | 19: Rubber packing |
| 100: Electrical signal | 100a: First electrical signal |
| 100b: Second electrical signal | 101a, 101b: First rectangular signal |
| 102a, 102b: Positive spike signal | 103a, 103b: Second Rectangular signal |
| 104a, 104b: Negative spike | |
| signal | |

What is claimed is:

1. A pain relief device comprising:
a generating unit that generates an electrical signal recognized as non-painful by the brain;
an electrode attached to at least one part of the body to deliver the generated electrical signal; and
a control unit that controls the operation of the generating unit and the electrode;
wherein, during the process in which a pain signal generated in a first region of the body is transmitted to the brain, the electrical signal delivered by the electrode is transmitted to the brain along with the pain signal through surface stimulus receptor of the first region, thereby alleviating the pain perceived by the brain in the first region,
wherein the electrical signal is an alternating current stimulation (ACS) signal, and
wherein the alternating current stimulation signal repeatedly switches on and off according to a predetermined first frequency, and is a first combined stimulation that applies the signal switched on according to the first frequency as stimulus according to a predetermined second frequency.

2. The pain relief device of claim 1, wherein the signal switched on according to the first frequency is a burst signal that a positive signal having a value exceeding a predetermined standard and a negative signal having a value below the predetermined standard repeat, and
each of the positive signal and the negative signal comprises at least one spike signal.

3. The pain relief device of claim 2, wherein the positive signal and negative signal consist of multiple positive spike signals and multiple negative spike signals.

4. The pain relief device of claim 2, wherein the burst signal is a signal comprising multiple rectangular signals having a positive value and a negative current value,
a first rectangular signal among the multiple rectangular signals comprises at least one positive spike signal, and
a second rectangular signal following the first rectangular signal comprises at least one negative spike signal.

5. The pain relief device of claim 4, wherein the control unit, when the degree of pain relief according to the first combined stimulation is below a predetermined range, controls the generating unit to generate a second combined stimulation signal that at least one of the first frequency, the second frequency, and the output, the waveform and the period of the stimulation according to the second frequency is modified, and
controls the second combined stimulation signal to be delivered to the surface stimulus receptor through the electrode.

6. The pain relief device of claim 2, wherein the electrical signal is a similar electrical signal within a certain error range of a biological signal in the body, thus being recognized as non-painful by the brain.

7. The pain relief device of claim 6, wherein the electrode is arranged in multiple numbers near the first region.

8. The pain relief device of claim 7, wherein the multiple electrodes deliver an electrical signal to promote ATP (adenosine triphosphate) generation in the first region, and the promoted ATP generation improves blood flow, thereby improving inflammation related to the first region.

9. The pain relief device of claim 8, wherein the electrical signal has an intensity of 100 μA to 1000 μA and is applied for 10 to 60 minutes.

10. The pain relief device of claim 1, wherein the pain occurring in the first region comprises knee joint pain, frozen shoulder pain, shoulder impingement syndrome pain, carpal tunnel syndrome pain, plantar fasciitis pain, rheumatoid arthritis pain, postherpetic neuralgia pain, back pain, lumbar stenosis pain, and neck pain.

11. A control method of a pain relief device comprising
a generating unit that generates an electrical signal recognized as non-painful by the brain;
an electrode attached to at least one part of the body to deliver the generated electrical signal; and
a control unit that controls the operation of the generating unit and the electrode;
wherein the control method comprising:
a) a first step of delivering the electrical signal to a surface stimulus receptor of a first region of the body;
b) a second step in which, during the process where a pain signal generated in the first region are transmitted to the brain, the electrical signal delivered to the surface stimulus receptor of the first region mix with the pain signal;
c) a third step in which the electrical signal and the pain signal are transmitted together to the brain; and
d) a fourth step in which the pain perceived by the brain in the first region is alleviated by the electrical signal,
wherein the electrical signal is an alternating current stimulation (ACS) signal, and
wherein the alternating current stimulation signal repeatedly switches on and off according to a predetermined first frequency, and is a first combined stimulation that applies the signal switched on according to the first frequency as stimuli according to a predetermined second frequency.

12. The control method of the pain relief device of claim 11, wherein the signal switched on according to the first frequency is a burst signal that a positive signal having a value exceeding a predetermined standard and a negative signal having a value below the predetermined standard repeat, and
each of the positive signal and the negative signal comprises at least one spike signal.

13. The control method of the pain relief device of claim 12, wherein the burst signal is a signal comprising multiple rectangular signals having a positive value and a negative current value,
a first rectangular signal among the multiple rectangular signals comprises at least one positive spike signal, and
a second rectangular signal following the first rectangular signals comprises at least one negative spike signal.

14. The control method of the pain relief device of claim 13, further comprising a fifth step in which, when the degree of pain relief according to the first combined stimulation in the fourth step is below a predetermined range, the control unit controls the generating unit to generate a second combined stimulation signal that at least one of the first frequency, the second frequency, and the output, the waveform and the period of the stimulation according to the second frequency is modified, and controls the second combined stimulation signal to be delivered to the surface stimulus receptor through the electrode.

15. The control method of the pain relief device of claim 12, wherein the electrical signal is a similar electrical signal within a certain error range of a biological signal in the body, thus being recognized as non-painful by the brain, and
the electrode is arranged in multiple numbers near the first region,
wherein the control method further comprises, after the fourth step:
a sixth step in which the multiple electrodes deliver a electrical signal to the surface stimulus receptor to promote adenosine triphosphate (ATP) generation in the first region; and
a seventh step in which, blood flow is improved based on the promoted ATP generation, thereby improving inflammation related to the first region.
